SEQUENCE LISTING (4 sequences)

SEQ ID NO: 1 - protein, 576 aa, Fomitopsis pinicola
ATTAEWQQRSIYQLVTDRFATSDGSSPACDTGDRVYCGGSWQGVINKLDYIQYMGFDAIWISPVVKNLEGSTGDGYSYHGYWAVDQNSVNEHFGTADDLNALSSALHARGMYLMVDVVVNHMAANTLPPDYSTFTPFSSESDFHTFCWITDYDNQTNVEQCWLGDSSVPLADCDTEADNVIDFFYNWIGELRANYTVDGFRIDTLKHVRQTFWPDFQTNAGVYAVGEVFDGDVNYVSPYTEVIDGVLDYPTYYQLTSAFESTSGSIQNLVDVIQSAQSTYSTMLFQVATFLENQDNPRFQSLTTDQGLVKNAMAWPFIADGIPILYYGQEQGYTGGNDPDNREALWLSGYEENKPLVQHARILNAARKAAIAASSSFLSTAVTFPSVGSNTLAASKYPLLSLLTNVGASGMPVWDVSSGTGYDEGTELIDALTCTTYTAGSSGSVSVTGSSGDPVILLPTSAYNASYCSELTGTDSTGSSDTVSVTFEVEYNTTYGENLYLTGSVSELVDWSVDDALLMSSADYPTWSLTVDLPPSTAIQYKYLTKYNGDVTWEDDPNNELTTPASGSVTQSDSWH

SEQ ID NO: 2 - protein, 593 aa, Fomitopsis pinicola
MWGSLLAASALVASALAATTAEWQQRSIYQLVTDRFATSDGSSPACDTGDRVYCGGSWQGVINKLDYIQYMGFDAIWISPVVKNLEGSTGDGYSYHGYWAVDQNSVNEHFGTADDLNALSSALHARGMYLMVDVVVNHMAANTLPPDYSTFTPFSSESDFHTFCWITDYDNQTNVEQCWLGDSSVPLADCDTEADNVIDFFYNWIGELRANYTVDGFRIDTLKHVRQTFWPDFQTNAGVYAVGEVFDGDVNYVSPYTEVIDGVLDYPTYYQLTSAFESTSGSIQNLVDVIQSAQSTYSTMLFQVATFLENQDNPRFQSLTTDQGLVKNAMAWPFIADGIPILYYGQEQGYTGGNDPDNREALWLSGYEENKPLVQHARILNAARKAAIAASSSFLSTAVTFPSVGSNTLAASKYPLLSLLTNVGASGMPVWDVSSGTGYDEGTELIDALTCTTYTAGSSGSVSVTGSSGDPVILLPTSAYNASYCSELTGTDSTGSSDTVSVTFEVEYNTTYGENLYLTGSVSELVDWSVDDALLMSSADYPTWSLTVDLPPSTAIQYKYLTKYNGDVTWEDDPNNELTTPASGSVTQSDSWH

SEQ ID NO: 3 - DNA, 1731 bp, Fomitopsis pinicola
gcgactacggcggaatggcagcaacgctcgatctaccagctcgtcacggatagattcgcgacctcggacggctcgtcgcccgcttgcgacaccggcgatcgcgtgtattgtggcgggtcgtggcagggggtcatcaataagctggattacatccagtacatgggcttcgacgccatctggatttcaccagtcgtgaagaacctagaaggcagcacgggagatggctactcgtaccatggatactgggcggtcgaccagaactcggtcaacgagcacttcggcactgcagacgatctgaatgcccttagcagcgcgttgcacgcgcgcgggatgtacctgatggtcgacgtcgtcgtgaaccacatggcggccaacacgctcccgccggactactcgaccttcacgccgttcagctccgagtcggacttccacaccttctgctggatcacggactacgacaaccagacgaacgtcgagcagtgctggctcggcgactcgagcgtgccgctcgcggactgcgacaccgaggctgacaacgtcatcgacttcttctacaactggatcggcgagctccgcgcgaactacactgtggatggtttcaggatcgacacgctgaagcatgtcaggcagacgttctggccagatttccagaccaacgcgggcgtgtacgccgtcggtgaggtctttgacggcgacgtgaactatgtttcgccctacactgaggttatcgacggagtgttggactacccgacgtactatcagctcacctccgccttcgagtccaccagcggctcaattcagaacctcgtcgacgtgattcagtctgcgcagtccacttactccacgatgctcttccaagtcgcgacgttcctcgagaaccaggacaacccgcggttccagagtcttaccactgatcaaggcctagtgaagaacgcgatggcgtggccgtttatcgcggatggcatccccattctttactacggtcaggagcaaggctacactggcggcaacgaccccgataaccgtgaagcgctgtggctgtccggatacgaggaaaacaagcctctcgtgcagcacgcccgcatcctcaacgctgcccgcaaggccgccatcgccgccagcagcagcttcctctccaccgccgtgaccttcccgtcggtgggcagcaacacgctcgccgcgtccaaatacccgctgctctcgctcctgaccaacgtaggcgcaagcggcatgcccgtttgggacgtctcctcgggaacgggatacgacgagggcacggagctgattgatgcgctcacgtgcacgacgtacaccgctgggagcagcggcagcgtgagcgtcacgggcagtagcggggaccccgtcatcctgctcccgacgagcgcatacaacgcgtcgtactgcagcgagctgacgggcacggactcgaccggcagctcggacacggtgtcggtgacgttcgaggtggagtacaacacgacgtacggcgagaatttgtatctcaccggctccgtctctgagctcgtggactggtccgtcgatgacgcactccttatgtcgtccgccgactacccgacctggagcctgacggtggacctccccccgagcacggcgatccagtacaagtatctgacgaagtacaatggcgacgtcacgtgggaggacgaccccaacaacgagctcacgacgcctgcgagcggctccgtcacgcagagcgacagctggcattga

SEQ ID NO: 4 - DNA, 1782 bp, Fomitopsis pinicola
atgtggggcagccttctcgcagcctctgccctcgtggcttccgcgcttgctgcgactacggcggaatggcagcaacgctcgatctaccagctcgtcacggatagattcgcgacctcggacggctcgtcgcccgcttgcgacaccggcgatcgcgtgtattgtggcgggtcgtggcagggggtcatcaataagctggattacatccagtacatgggcttcgacgccatctggatttcaccagtcgtgaagaacctagaaggcagcacgggagatggctactcgtaccatggatactgggcggtcgaccagaactcggtcaacgagcacttcggcactgcagacgatctgaatgcccttagcagcgcgttgcacgcgcgcgggatgtacctgatggtcgacgtcgtcgtgaaccacatggcggccaacacgctcccgccggactactcgaccttcacgccgttcagctccgagtcggacttccacaccttctgctggatcacggactacgacaaccagacgaacgtcgagcagtgctggctcggcgactcgagcgtgccgctcgcggactgcgacaccgaggctgacaacgtcatcgacttcttctacaactggatcggcgagctccgcgcgaactacactgtggatggtttcaggatcgacacgctgaagcatgtcaggcagacgttctggccagatttccagaccaacgcgggcgtgtacgccgtcggtgaggtctttgacggcgacgtgaactatgtttcgccctacactgaggttatcgacggagtgttggactacccgacgtactatcagctcacctccgccttcgagtccaccagcggctcaattcagaacctcgtcgacgtgattcagtctgcgcagtccacttactccacgatgctcttccaagtcgcgacgttcctcgagaaccaggacaacccgcggttccagagtcttaccactgatcaaggcctagtgaagaacgcgatggcgtggccgtttatcgcggatggcatccccattctttactacggtcaggagcaaggctacactggcggcaacgaccccgataaccgtgaagcgctgtggctgtccggatacgaggaaaacaagcctctcgtgcagcacgcccgcatcctcaacgctgcccgcaaggccgccatcgccgccagcagcagcttcctctccaccgccgtgaccttcccgtcggtgggcagcaacacgctcgccgcgtccaaatacccgctgctctcgctcctgaccaacgtaggcgcaagcggcatgcccgtttgggacgtctcctcgggaacgggatacgacgagggcacggagctgattgatgcgctcacgtgcacgacgtacaccgctgggagcagcggcagcgtgagcgtcacgggcagtagcggggaccccgtcatcctgctcccgacgagcgcatacaacgcgtcgtactgcagcgagctgacgggcacggactcgaccggcagctcggacacggtgtcggtgacgttcgaggtggagtacaacacgacgtacggcgagaatttgtatctcaccggctccgtctctgagctcgtggactggtccgtcgatgacgcactccttatgtcgtccgccgactacccgacctggagcctgacggtggacctccccccgagcacggcgatccagtacaagtatctgacgaagtacaatggcgacgtcacgtgggaggacgaccccaacaacgagctcacgacgcctgcgagcggctccgtcacgcagagcgacagctggcattga

(19) United States
(12) Patent Application Publication
(10) Pub. No.: US 2021/0071160 A1
(43) Pub. Date: Mar. 11, 2021

(54) IMPROVED WASHING PERFORMANCE USING A NOVEL ALPHA-AMYLASE FROM FOMITOPSIS PINICOLA (FPI)

(71) Applicant: Henkel AG & Co. KGaA, Duesseldorf (DE)

(72) Inventors: Nina MUSSMANN, Willich (DE); Susanne WIELAND, Zons/Dormagen (DE); Inken PRUESER, Duesseldorf (DE); Margret VAN LIER, Hilden (DE); Christian DEGERING, Erkrath (DE); Ralf G. BERGER, Hannover (DE); Florian DOERING, Penzberg (DE)

(73) Assignee: Henkel AG & Co. KGaA, Duesseldorf (DE)

(21) Appl. No.: 16/645,219

(22) PCT Filed: May 22, 2019
(86) PCT No.: PCT/EP2019/063168

§ 371 Date: Mar. 6, 2020

(30) Foreign Application Priority Data
May 29, 2018 (DE) 10 2018 208 445.9

(51) Int. Cl. C12N 9/26; C11D 3/386

(52) U.S. Cl. CPC C12N 9/2414; C11D 3/386; C12Y 302/01001

(57) ABSTRACT

The present disclosure lies in the field of enzyme technology. The present disclosure relates to amylases which may be used in particular with regard to use in detergents and cleaning products, all sufficiently similar amylases with a correspondingly similar sequence according to SEQ ID NO. 1 or SEQ ID NO. 2 and the nucleic acids coding them. The present disclosure further relates to their preparation as well as the method for using these amylases, their use as such and products containing them, in particular detergents and cleaning products.

### CROSS-REFERENCE TO RELATED APPLICATION

This application is a U.S. National-Stage entry under 35 U.S.C. § 371 based on International Application No. PCT/EP2019/063168, filed May 22, 2019, which was published under PCT Article 21(2) and which claims priority to German Application No. 10 2018 208 445.9, filed May 29, 2018, which are all hereby incorporated in their entirety by reference.

### TECHNICAL FIELD

The present disclosure concerns the field of enzyme technology. The present disclosure relates to amylases, which may be used in particular with regard to use in detergents and cleaning agents, all sufficiently similar amylases with a correspondingly similar sequence according to SEQ ID No. 1 or SEQ ID No. 2 and nucleic acids coding for them. The present disclosure further concerns their preparation as well as processes for the use of these amylases, their use as such and products containing them, in particular detergents and cleaning agents.

### BACKGROUND

Amylases belong to the technically important enzymes. Their use for detergents and cleaning agents is industrially established and they are typically found in modern, high-performance detergents and cleaning agents. An amylase is an enzyme that catalyzes the hydrolysis of the inner α-(1-4)-glycoside bonds of the amylose, but not the cleavage of terminal or α-(1-6)-glycoside bonds. Amylases therefore represent a group of esterases (E.C. 3.2.1.1.). Amylases catalyze the cleavage of starch, glycogen and other oligo- and polysaccharides which have an α-(1-4)-glycoside bond. In this respect, amylases act against starch residues in laundry and catalyze their hydrolysis (endohydrolysis). Amylases with broad substrate spectra are used in particular where inhomogeneous raw materials or substrate mixtures have to be converted, e.g. in detergents and cleaning agents, since soiling may consist of differently structured starch molecules and oligosaccharides. Amylases used in detergents or cleaning agents known from the state of the art are usually of microbial origin and are usually derived from bacteria or fungi, for example of the genera Bacillus, Pseudomonas, Acinetobacter, Micrococcus, Humicola, Trichoderma or Trichosporon, especially Bacillus. Amylases are usually produced by suitable microorganisms using biotechnological methods known per se, for example, by transgenic expression hosts of the genera Bacillus or by filamentous fungi.

U.S. Pat. No. 8,512,986 discloses amylase and its use in a starch liquefaction process in which starch is degraded to small oligo- and/or polysaccharide fragments. U.S. Pat. Nos. 7,407,677 B2 and 8,852,912 B2 also disclose specific amylases and their fragments for use in detergents and cleaning agents.

Nevertheless, there is still a need for amylase variants with modified biochemical properties that provide improved performance in industrial applications.

### BRIEF SUMMARY

Amylases, methods for producing amylases, and detergent and cleaning products containing such amylases are provided herein. In an exemplary embodiment, an amylase includes an amino acid sequence having at least about 70% sequence identity to SEQ ID NO. 1 or SEQ ID NO. 2 over its entire length.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now been found that an amylase from Basidiomyceta, in particular Fomitopsis pinicola (Fpi), or an amylase sufficiently similar (in terms of sequence identity) to it, is particularly suitable for use in detergents or cleaning agents, as it hydrolyzes a wide range of starch substrates under standard washing conditions.

The subject of the present disclosure is therefore an amylase comprising an amino acid sequence which has at least about 70% sequence identity with the amino acid sequence given in SEQ ID NO. 1 or SEQ ID NO. 2 over its entire length.

The subject-matter of the present disclosure is also an amylase comprising an amino acid sequence which has at least about 70% sequence identity with the amino acid sequence given in SEQ ID NO. 1 or SEQ ID NO. 2 over its entire length, or variants thereof. The variants as contemplated herein are obtainable from an amylase which has an amino acid sequence with at least about 70% sequence identity with the amino acid sequence indicated in SEQ ID NO. 1 or SEQ ID NO. 2 over its entire length as starting molecule by single or multiple conservative amino acid substitution. Alternatively or in addition, the variants as contemplated herein are obtainable from an amylase which has an amino acid sequence with at least about 70% sequence identity with the amino acid sequence indicated in SEQ ID NO. 1 or SEQ ID NO. 2 over its entire length, as starting molecule by fragmentation, deletion, insertion or substitution mutagenesis and an amino acid sequence which is identical to the starting molecule over a length of at least about 403, about 410, about 420, about 430, about 440, about 450, about 460, about 470, about 480, about 490, about 500, about 510, about 520, about 530, about 540, about 550, about 560, about 565, about 566, about 567, about 568, about 569, about 570, about 571, about 572, about 573, about 574, about 575 or about 576 contiguous amino acids.

A further subject-matter of the present disclosure is a process for producing an amylase, comprising providing a starting amylase having at least about 70% sequence identity to the amino acid sequence given in SEQ ID NO. 1 SEQ ID NO. 2 over its entire length, or variants of the starting amylase, wherein the variants are as defined above.

An amylase within the meaning of the present patent application therefore comprises both the amylase as such and an amylase produced by a process as contemplated herein. All statements on amylase therefore refer both to the amylase as a substance and to the corresponding processes, in particular production processes of the amylase. A nucleotide sequence corresponding to the amino acid sequence according to SEQ ID NO. 1 or SEQ ID NO. 2 is given in SEQ ID NO. 3 or SEQ ID NO. 4.

As further subject-matters of present disclosure, nucleic acids encoding the amylases of the present disclosure or the production methods for amylases of the present disclosure for these amylases, non-human host cells containing the amylases of the present disclosure or nucleic acids containing nucleic acids are associated with.

The present disclosure also relates to products comprising amylases, in particular detergents, washing and cleaning processes, and uses defined by the amylases described herein, wherein the amylases used here have at least about 70% sequence identity with the amino acid sequence given in SEQ ID NO. 1 or SEQ ID NO. 2 over their entire length or are variants thereof. The variants used here are obtainable from an amylase which has an amino acid sequence with at least about 70% sequence identity with the amino acid sequence indicated in SEQ ID NO. 1 or SEQ ID NO. 2 over its entire length as the starting molecule by single or multiple conservative amino acid substitution. Alternatively or in addition, the variants used are obtained from an amylase which has an amino acid sequence with at least about 70% sequence identity with the amino acid sequence indicated in SEQ ID NO. 1 or SEQ ID NO. 2 over its entire length, as starting molecule by fragmentation, deletion, insertion or substitution mutagenesis and comprises an amino acid sequence, which is identical to the parent molecule over a length of at least about 403, about 410, about 420, about 430, about 440, about 450, about 460, about 470, about 480, about 490, about 500, about 510, about 520, about 530, about 540, about 550, about 560, about 565, about 566, about 567, about 568, about 569, about 570, about 571, about 572, about 573, about 574, about 575 or about 576 contiguous amino acids.

The present disclosure is based on the inventors' surprising finding that an amylase from Basidiomyceta, in particular Fomitopsis pinicola, which comprises an amino acid sequence which is at least about 70% identical to the amino acid sequence given in SEQ ID NO. 1 or SEQ ID NO. 2, causes hydrolysis of a broad spectrum of starch substrates under standard washing conditions. This is particularly surprising in that none of the amylases from Basidiomyceta, in particular Fomitopsis pinicola, has been described for use in detergents or cleaning agents.

The amylases as contemplated herein have a high stability in detergents or cleaning agents, for example against surfactants and/or bleaching agents and/or against temperature influences and/or against acidic or alkaline conditions and/or against changes in pH value and/or against denaturing or oxidizing agents and/or against proteolytic degradation and/or against a change in redox ratios. Consequently, performance-enhanced amylase variants are provided with particularly preferred embodiments of the present disclosure. Such advantageous versions of amylases as contemplated herein thus allow improved washing results on starch-containing stains in a wide temperature range.

An amylase as contemplated herein has an enzymatic activity, i.e. it is capable of hydrolyzing starch and oligosaccharides, especially in a detergent or cleaning agent. An amylase as contemplated herein is therefore an enzyme which catalyzes the hydrolysis of α-(1-4)-glycoside bonds in glycoside substrates and is thus capable of cleaving starch or oligosaccharides. Furthermore, an amylase as contemplated herein is preferably a mature amylase, i.e. the catalytically active molecule without signal and/or propeptide(s). Unless otherwise stated, the sequences given also refer to mature (processed) enzymes. As used herein, SEQ ID NO. 1 is the amino acid sequence of the mature protein, SEQ ID NO. 2 indicates the sequence including signal peptide(s).

“Amylase as contemplated herein” as used herein, refers to amylases containing at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99%, about 99.1%, about 99.2%, about 99.3%, about 99.4%, about 99.5%, about 99.6%, about 99.7%, about 99.8% or about 99.9% sequence identity with the sequence identified in SEQ ID NO. 1 or SEQ ID NO. 2 over the entire length or are variants thereof. The variants are obtainable from an amylase with the indicated sequence identity as the starting molecule by single or multiple conservative amino acid substitution. Alternatively or additionally, the variants used are obtainable from an amylase with the indicated sequence identity as starting molecule by fragmentation, deletion, insertion or substitution mutagenesis and comprises an amino acid sequence, which is identical to the parent molecule over a length of at least about 403, about 410, about 420, about 430, about 440, about 450, about 460, about 470, about 480, about 490, about 500, about 510, about 520, about 530, about 540, about 550, about 560, about 565, about 566, about 567, about 568, about 569, about 570, about 571, about 572, about 573, about 574, about 575 or about 576 contiguous amino acids.

In various embodiments of the present disclosure, the amylase comprises an amino acid sequence which is at least about 95%, about 95.5%, about 96%, about 96.5%, about 97%, about 97.5%, about 98%, about 98.5%, about 98.8%, about 99.0%, about 99.2%, about 99.4%, about 99.5%, about 99.6% or about 99.8% identical to the amino acid sequence given in SEQ ID NO. 1 over its entire length.

In further various embodiments of the present disclosure, amylase is a freely present enzyme. This means that the amylase may act directly with all components of a product and, if the product is a liquid agent, that the amylase is directly in contact with the solvent of the product as contemplated herein (e.g. water). In further embodiments, the amylase of the present disclosure in a product may form an interaction complex with other molecules or contain an “envelope”. Here, a single or several amylase molecules may be separated from the other components of a product by a structure surrounding them. Such a separating structure may be created by, but is not limited to, vesicles, such as a micelle or liposome. The surrounding structure may also be a virus particle, a bacterial cell or a eukaryotic cell. In various embodiments, the amylase as contemplated herein may be present in cells of Basidiomyceta expressing this amylase or in cell culture supernatants of such cells.

The identity of nucleic acid or amino acid sequences is determined by sequence comparison. This sequence comparison is based on the BLAST algorithm, which is established in the state of the art and commonly used (see for example Altschul, S. F., Gish, W., Miller, W., Myers, E. W. & Lipman, D. J. (1990): “Basic local alignment search tool”, J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”, Nucleic Acids Res. 25:3389-3402) and in principle it is done by assigning similar sequences of nucleotides or amino acids in the nucleic acid or amino acid sequences to each other. A tabular mapping of the respective positions is called alignment. Another state-of-the-art algorithm is the FASTA algorithm. Sequence comparisons (alignments), in particular multiple sequence comparisons, are created with computer programs. The Clustal series (see for example Chenna et al. (2003): “Multiple sequence alignment with the Clustal series of programs”, Nucleic Acid Res. 31:3497-3500), T-Coffee (see for example Notredame et al. (2000): “T-Coffee: A novel method for multiple sequence alignments”, J. Mol. Biol. 302:205-217) or programs based on these programs or algorithms Sequence comparisons (alignments) are also possible with the computer program Vector NTI® Suite 10.3 (Invitrogen Corporation, 1600 Faraday Avenue, Carlsbad, Calif., USA) with the specified standard parameters, whose AlignX module for sequence comparisons is based on ClustalW.

Such a comparison also allows a statement about the similarity of the compared sequences to each other. It is usually expressed in percent identity, i.e. the proportion of identical nucleotides or amino acid residues at the same positions or in an alignment corresponding to each other. In the case of amino acid sequences, the broader term homology includes conserved amino acid exchanges, i.e. amino acids with similar chemical activity, since these usually have similar chemical activities within the protein. Therefore, the similarity of the compared sequences may also be expressed as percentage homology or percentage similarity. Identity and/or homology information may be given for entire polypeptides or genes or only for individual regions. Homologous or identical regions of different nucleic acid or amino acid sequences are therefore defined by matches in the sequences. Such regions often have identical functions. They may be small and comprise only a few nucleotides or amino acids. Often such small regions perform essential functions for the overall activity of the protein. It may therefore be useful to relate sequence matches only to individual, possibly small, regions. However, unless otherwise indicated, identity or homology information in the present application refers to the total length of the nucleic acid or amino acid sequence indicated.

In the context of the present disclosure, the indication that an amino acid position corresponds to a numerically designated position in SEQ ID NO. 1 therefore means that the corresponding position is assigned to the numerically designated position in SEQ ID NO. 1 in an alignment as defined above.

In a further embodiment of the present disclosure, the amylase cleaning performance is not significantly reduced compared to that of an amylase comprising an amino acid sequence corresponding to the amino acid sequences given in SEQ ID NO. 1, i.e. it has at least about 70%, about 75%, about 80%, about 85%, about 90%, about 95% of the reference washing performance The cleaning performance may be determined in a washing system containing a detergent at a dosage of between about 4.5 and about 7.0 grams per liter of washing liquor and the amylase, the amylases to be compared being used at the same concentration (relative to active protein) and the cleaning performance being determined against soiling on cotton by measuring the degree of cleaning of the washed fabrics. For example, the washing process may be carried out for about 60 minutes at a temperature of about 40° C. and the water may have a water hardness of between about 5° and about 25°, preferably about 10° and about 20°, more preferably about 13° and about 17° and further preferably about 15.5° and about 16.5° (German hardness). The concentration of amylase in the detergent intended for this washing system is from about 0.001 to about 1% by weight, preferably from about 0.001 to about 0.1% by weight, and even more preferably from about 0.01 to about 0.06% by weight, based on active purified protein.

A preferred liquid detergent for such a washing system is composed as follows (all figures in % by weight): about 7% alkylbenzene sulfonic acid, about 9% anionic surfactants, about 4% Na salts of C₁₂-C₁₈ fatty acids, about 7% non-ionic surfactants, about 0.7% phosphonates, about 3.2% citric acid, about 3.0% NaOH, about 0.04% defoamer, about 5.7% 1,2-propanediol, about 0.1% preservatives, about 2% ethanol, about 0.2% colorant transfer inhibitor, balance demineralized water. Preferably, the dosage of the liquid detergent is between about 4.5 and about 6.0 grams per liter of washing liquor, for example about 4.7, about 4.9 or about 5.9 grams per liter of washing liquor. Preferably, washing is done in a pH value range between about pH 7.5 and about pH 10.5, preferably between about pH 7.5 and about pH 9.

In the context of the present disclosure, the determination of the cleaning performance is carried out at about 40° C. using a liquid detergent as indicated above, the washing process preferably being carried out for about 60 minutes.

The degree of whiteness, i.e. the lightening of the soiling, as a measure of the cleaning performance is determined by optical measuring methods, preferably photometrically. A suitable device for this purpose is, for example, the Minolta CM508d spectrometer. Usually the devices used for the measurement are calibrated beforehand with a white standard, preferably a white standard supplied with the device.

By using the respective amylase with the same activity, it is ensured that even if the ratio of active substance to total protein (the values of the specific activity) diverges, the respective enzymatic properties, e.g. the cleaning performance on certain soiling, are compared. In general, a low specific activity may be compensated by adding a larger amount of protein.

The amylase activity is determined in the usual way, preferably by an optical measuring method, preferably a photometric method. The test suitable for this purpose comprises the amylase-dependent cleavage of the substrate para-nitrophenyl maltoheptaoside. This is cleaved by the amylase into para-nitrophenyl oligosaccharide. The para-nitrophenyl oligosaccharide is in turn catalyzed by the enzymes glucoamylase and alpha-glucosidase to glucose and para-nitrophenol. The presence of para-nitrophenol may be determined using a photometer, e.g. the Tecan Sunrise device and XFLUOR software, at 405 nm and thus allows conclusions to be drawn about the enzymatic activity of the amylase.

The protein concentration may be determined by known methods, for example the BCA method (bicinchoninic acid; 2,2′-biquinolyl-4,4′-dicarboxylic acid) or the biuret method (A. G. Gornall, C. S. Bardawill and M. M. David, J. Biol. Chem., 177 (1948), pp. 751-766). In this respect, the determination of the active protein concentration may be carried out by titration of the active centers using a suitable irreversible inhibitor and determination of the residual activity (see M. Bender et al., J. Am. Chem. Soc. 88, 24 (1966), pp. 5890-5913).

Proteins may be combined into groups of immunologically related proteins by reaction with an antiserum or a specific antibody. The members of such a group are exemplified by the fact that they share the same antigenic determinant recognized by an antibody. They are therefore structurally similar to each other to such an extent that they are recognized by an antiserum or certain antibodies. A further subject-matter of present disclosure is therefore formed by amylases which have at least one and increasingly preferably two, three or four identical antigenic determinants with an amylase as contemplated herein. Such amylases are structurally so similar to the amylases as contemplated herein due to their immunological similarities that a similar function may also be assumed.

Amylases as contemplated herein may exhibit further amino acid changes, in particular amino acid substitutions, insertions or deletions, in comparison to the amylase described in SEQ ID NO. 1 or SEQ ID NO 2. Such amylases are, for example, further developed by specific genetic modification, i.e. by mutagenesis methods, and optimized for certain applications or with regard to specific properties (for example with regard to their catalytic activity, stability, etc.). Furthermore, nucleic acids that are the object of the present disclosure may be introduced into recombination approaches and thus be used to produce completely novel amylases or other polypeptides.

The aim is to introduce targeted mutations such as substitutions, insertions or deletions into known molecules, for example, to improve the cleaning performance of enzymes as contemplated herein. For this purpose, in particular the surface charges and/or the isoelectric point of the molecules and thus their interaction with the substrate may be altered. For example, the net charge of the enzymes may be altered in order to influence substrate binding, especially for use in detergents and cleaning agents. Alternatively or as a supplement, the stability of the amylase may be further increased by one or several corresponding mutations, thereby improving its cleaning performance. Advantageous properties of individual mutations, e.g. individual substitutions, may complement each other. An amylase which has already been optimized with respect to certain properties, for example with respect to its activity and/or its tolerance with respect to the substrate spectrum, may therefore be further developed within the scope of the present disclosure.

For the description of substitutions that affect exactly one amino acid position (amino acid exchanges), the following convention is applied: first the naturally existing amino acid is designated in the form of the internationally used one-letter code, then the corresponding sequence position follows, and finally the inserted amino acid. Several exchanges within the same polypeptide chain are separated by slashes. For insertions, additional amino acids are named after the sequence position. In deletions, the missing amino acid is replaced by a symbol, for example an asterisk or a dash, or a A is given before the corresponding position. For example, R45Q describes the substitution of arginine at position 45 by glutamine, N45AQ describes the insertion of glutamine after the amino acid alanine at position 45 and N45* or AN45 describes the deletion of asparagine at position 45. This nomenclature is known to experts in the field of enzyme technology.

A further object of the present disclosure is therefore an amylase, exemplified in that it is obtainable from an amylase as described above as a starting molecule by single or multiple conservative amino acid substitution. The term “conservative amino acid substitution” means the exchange (substitution) of one amino acid residue with another amino acid residue, whereby this exchange does not lead to a change in polarity or charge at the position of the exchanged amino acid, e.g. the exchange of a non-polar amino acid residue with another non-polar amino acid residue. Conservative amino acid substitutions within the scope of the present disclosure include, for example: G=A=S, I=V=L=M, D=E, N=Q, K=R, Y=F, S=T, G=A=I=V=L=M=Y=F=W=P=S=T. In this context, the amylase may comprise, before and, for example, also after conservative amino acid substitution, an amino acid sequence of the amino acid sequence indicated in SEQ ID NO. 1 or SEQ ID NO. 2 over its entire length which is at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9% identical.

Alternatively or in addition, the amylase is obtainable from an amylase as described above as the starting molecule by fragmentation, deletion, insertion or substitution mutagenesis and comprises an amino acid sequence which, over a length of at least about 403, about 410, about 420, about 430, about 440, about 450, about 460, about 470, about 480, about 490, about 500, about 510, about 520, about 530, about 540, about 550, about 560, about 565, about 566, about 567, about 568, about 569, about 570, about 571, about 572, about 573, about 574, about 575 or about 576 contiguous amino acids is identical with the starting molecule. In this context, the amylase before and, for example, also after fragmentation, deletion, insertion or substitution mutagenesis, may comprise an amino acid sequence which is identical with the amino acid sequence indicated in SEQ ID NO. 1 or SEQ ID NO. 2 over its entire length, at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9%.

For example, it is possible to delete individual amino acids at the termini or in the loops of the enzyme without losing or reducing the catalytic activity. Furthermore, such fragmentation, deletion, insertion or substitution mutagenesis may, for example, also reduce the allergenicity of enzymes and thus improve their overall applicability. It is advantageous for the enzymes to retain their catalytic activity even after mutagenesis, i.e. their catalytic activity corresponds at least to that of the starting enzyme, i.e. in a preferred embodiment the catalytic activity is at least about 80%, preferably at least about 90% of the activity of the starting enzyme. Other substitutions may also show beneficial effects. Both single and several related amino acids may be substituted by other amino acids.

The other amino acid positions are defined here by aligning the amino acid sequence of an amylase as contemplated herein with the amino acid sequence of the amylase from Basidiomyceta, in particular Fomitopsis pinicola, as indicated in SEQ ID NO 1. or SEQ ID NO 2. Furthermore, the assignment of the positions is based on the mature protein. This assignment is also to be used in particular if the amino acid sequence of an amylase as contemplated herein comprises a higher number of amino acid residues than the amylase from Basidiomyceta, in particular Fomitopsis pinicola, as indicated in SEQ ID NO. 1 or SEQ ID NO. 2. Starting from the said positions in the amino acid sequence of the amylase from Basidiomyceta, in particular Fomitopsis pinicola, the positions of alteration in an amylase as contemplated herein are those which are assigned to these very positions in an alignment.

Further confirmation of the correct assignment of the amino acids to be altered, i.e. in particular their functional correspondence, may be provided by comparative experiments in which the two positions assigned to each other on the basis of an alignment are altered in the same way in both amylases compared with each other and it is observed whether the enzymatic activity is altered in the same way in both. If, for example, an amino acid exchange in a specific position of the amylase from Basidiomyceta, in particular Fomitopsis pinicola, according to SEQ ID NO. 1 or SEQ ID NO. 2 is accompanied by an alteration of an enzymatic parameter, for example, an increase in the KM value, and if a corresponding alteration of the enzymatic parameter, for example, also an increase in the KM value, is observed in an amylase variant as contemplated herein, the amino acid exchange of which has been achieved by the same introduced amino acid, this is to be regarded as confirmation of the correct assignment.

In particular, as contemplated herein, fragments of amylase as defined herein are also included, in particular those according to SEQ ID NO. 1 which are shortened at the N and/or C terminus in such a way that one or several amino acids of the amylase, for example about 1, about 2, about 3, about 4, about 5, about 6, about 7, about 8, about 9 or about 10, are no longer contained. Variants of these shortened fragments may also be used in various embodiments of the present disclosure, which are identical to the variant based on the amino acid sequence set out in SEQ ID NO. 1, shortened by (in each case) from about 1 to about 10 N terminal and/or C terminal amino acids over the total length to at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9%.

For example, as contemplated herein, amylases are also detected which have an amino acid sequence which, beyond the amylase, comprise an amino acid sequence which has at least about 70%, preferably at least about 80%, particularly preferably at least about 95% sequence identity with the amino acid sequence given in SEQ ID NO. 1 over its entire length or the variants thereof described herein, without the catalytic activity being lost or reduced thereby. Preferably, such amylases are those which have N- and/or C-terminally additional amino acids, for example the signal peptide or fragments of the signal peptide, the signal peptide or the fragments of the signal peptide being formed during the production of the amylase.

As contemplated herein, amylases are also detected which have an amino acid sequence which, compared with an amylase comprising an amino acid sequence which has at least about 70%, preferably at least about 80%, particularly preferably at least 95% sequence identity with the amino acid sequence given in SEQ ID NO. 2 and/or the variants thereof described herein, are shortened at the N-terminus in such a way that the signal peptide or one or several of the amino acids of the signal peptide, for example about 1, about 2, about 3, about 4, about 5, about 6, about 7, about 8, about 9, about 10, about 11, about 12, about 13, about 14, about 15, about 16 or about 17, in particular the N-terminal 17 amino acids, are no longer present. Variants of these amylases with the signal peptide or fragments of the signal peptide may also be used in various embodiments of the present disclosure which are shorter than the amino acid sequence given in SEQ ID NO. 2 by 1 to 17 N-terminal amino acids over the total length by at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9%.

All of the facts mentioned are also applicable to the methods for the production of an amylase as contemplated herein.

Accordingly, a method as contemplated herein comprises a method for producing an amylase, comprising the provision of a starting amylase which has at least about 70%, preferably at least about 80%, particularly preferably at least about 95% sequence identity to the amino acid sequence indicated in SEQ ID NO. 1 or SEQ ID NO. 2 over its entire length, or variants of the starting amylase, wherein the method as contemplated herein for producing the variants comprises, for example, one or several of the following method steps:
a) inserting a single or multiple conservative amino acid substitution into a starting amylase according to SEQ ID NO. 1 or SED ID NO. 2;
b) altering the amino acid substitution set forth in SEQ ID NO. 1 or SED ID NO. 2 by fragmentation, deletion, insertion or substitution mutagenesis, such that the amylase comprises an amino acid sequence which, over a length of at least about 403, about 410, about 420, about 430, about 440, about 450, about 460, about 470, about 480, about 490, about 500, about 510, about 520, about 530, about 540, about 550, about 560, about 565, about 566, about 567, about 568, about 569, about 570, about 571, about 572, about 573, about 574, about 575 or about 576 contiguous amino acids is identical to the starting molecule.

All embodiments also apply to the method as contemplated herein.

In further embodiments of the present disclosure, the amylase or the amylase produced by a method as contemplated herein is still at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94,5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9% identical with the amino acid sequence indicated in SEQ ID NO. 1 over its entire length.

A further subject-matter of the present disclosure is a previously described amylase which is additionally stabilized, in particular by one or several mutations, for example substitutions, or by coupling to a polymer. An increase in stability during storage and/or during use, for example, during the washing process, results in the enzymatic activity lasting longer and thus improving the cleaning performance In principle, all stabilization options described and/or appropriate in the state of the art are possible. Preference is given to those stabilizations that are achieved by mutations of the enzyme itself, since such stabilizations do not require any further processing steps after the enzyme has been obtained. Examples of suitable sequence modifications are given above. Other suitable sequence modifications are known from the state of the art.

Examples of stabilization are:
protection against the influence of denaturing agents such as surfactants by mutations that cause an alteration of the amino acid sequence on or at the surface of the protein;
exchange of amino acids that are close to the N-terminus for those that are likely to contact the rest of the molecule via non-covalent interactions, thus contributing to the maintenance of the globular structure.

Preferred embodiments are those in which the enzyme is stabilized in several ways, since several stabilizing mutations act additively or synergistically.

A further subject-matter of the present disclosure is an amylase as described above, exemplified in that it has at least one chemical modification. An amylase with such a modification is called a derivative, i.e. the amylase is derivatized.

For the purposes of the present application, derivatives are therefore proteins whose pure amino acid chain has been chemically modified. Such derivatizations may, for example, be carried out in vivo by the host cell that expresses the protein. In this respect, couplings of low-molecular compounds such as lipids or oligosaccharides are particularly noteworthy. Derivatizations may also be carried out in vitro, for example by chemical conversion of a side chain of an amino acid or by covalent binding of another compound to the protein. For example, the coupling of amines to carboxyl groups of an enzyme is possible to change the isoelectric point. Such other compound may also be a further protein which is bound to a protein as contemplated herein, for example, via bifunctional chemical compounds. Derivatization is also understood to mean covalent binding to a macromolecular carrier, or non-covalent inclusion in suitable macromolecular cage structures. Derivatizations may, for example, influence the substrate specificity or the binding strength to the substrate or cause a temporary blocking of the enzymatic activity if the coupled substance is an inhibitor. This may be useful, for example, for the period of storage. Such modifications may also affect stability or enzymatic activity. They may also serve to reduce the allergenicity and/or immunogenicity of the protein and thus, for example, increase its skin tolerance. For example, couplings with macromolecular compounds, such as polyethylene glycol, may improve the stability and/or skin tolerance of the protein.

Derivatives of a protein as contemplated herein may also be understood in the broadest sense as preparations of these proteins. Depending on the extraction, processing or preparation, a protein may be associated with various other substances, for example, from the culture of the producing microorganisms. A protein may also have been specifically mixed with other substances, for example, to increase its storage stability. As contemplated herein, therefore, all preparations are also a protein as contemplated herein. This is also independent of whether or not it actually develops this enzymatic activity in a particular preparation. For it may be desired that it has no or only little activity during storage and only develops its enzymatic function at the time of use. This may be controlled, for example, by means of appropriate accompanying substances. In particular, the joint preparation of amylases with specific inhibitors is possible in this respect.

With regard to all the amylases or amylase variants and/or derivatives described above, those whose catalytic activity and/or substrate tolerance corresponds to that of the amylase according to SEQ ID NO. 1 are particularly preferred in the context of the present disclosure, the catalytic activity and the substrate tolerance being determined as described above.

A further subject-matter of the present disclosure is a nucleic acid coding for an amylase as contemplated herein, as well as a vector containing such a nucleic acid, in particular a cloning vector or an expression vector. In preferred embodiments, the nucleic acid is a nucleic acid according to SEQ ID NO. 3 or SEQ ID NO. 4. Accordingly, a particularly preferred vector as contemplated herein is a vector comprising a nucleic acid according to SEQ ID NO. 3 or SEQ ID NO. 4.

These may be DNA or RNA molecules. They may be present as a single strand, as a single strand complementary to this single strand or as a double strand. Especially in the case of DNA molecules, the sequences of both complementary strands must be considered in all three possible reading frames. It should also be taken into account that different codons, i.e. base triplets, may code for the same amino acids, so that a particular amino acid sequence may be encoded by several different nucleic acids. Due to this degeneracy of the genetic code, all nucleic acid sequences which may encode one of the amylases described above are included in this subject-matter of the present disclosure. The expert is able to determine these nucleic acid sequences without any doubt, because despite the degeneracy of the genetic code, defined amino acids may be assigned to individual codons. Therefore, the expert may easily determine nucleic acids coding for this amino acid sequence starting from an amino acid sequence. Furthermore, in nucleic acids as contemplated herein, one or several codons may be substituted by synonymous codons. This aspect refers in particular to the heterologous expression of the enzymes as contemplated herein. Thus, every organism, for example, a host cell of a production strain, has a certain codon use. Codon use means the translation of the genetic code into amino acids by the respective organism. Bottlenecks in protein biosynthesis may occur if the codons located on the nucleic acid are confronted with a comparatively small number of loaded tRNA molecules in the organism. Although coding for the same amino acid, this leads to a codon in the organism being translated less efficiently than a synonymous codon coding for the same amino acid. Due to the presence of a higher number of tRNA molecules for the synonymous codon, it may be translated more efficiently in the organism.

Using methods that are generally known today, such as chemical synthesis or polymerase chain reaction (PCR) in conjunction with standard molecular biological and/or protein chemical methods, it is possible for an expert to produce the corresponding nucleic acids up to complete genes using known DNA and/or amino acid sequences. Such methods are known, for example, from Sambrook, J., Fritsch, E. F. and Maniatis, T. 2001 Molecular cloning: a laboratory manual, 3rd edition Cold Spring Laboratory Press.

For the purpose of the present disclosure, vectors are elements consisting of nucleic acids which contain a nucleic acid as contemplated herein as the characteristic nucleic acid region. They are capable of establishing this nucleic acid as a stable genetic element in a species or cell line over several generations or cell divisions. Vectors are special plasmids, i.e. circular genetic elements, especially when used in bacteria. In the context of the present disclosure, a nucleic acid as contemplated herein is cloned into a vector. Vectors include, for example, those whose origins are bacterial plasmids, viruses or bacteriophages, or predominantly synthetic vectors or plasmids with elements of various origins. With the additional genetic elements present in each case, vectors are able to establish themselves as stable units in the host cells concerned over several generations. They may be present extrachromosomally as separate units or integrated into a chromosome or chromosomal DNA.

Expression vectors comprise nucleic acid sequences which enable them to replicate in the host cells containing them, preferably microorganisms, particularly preferably bacteria, and to cause a contained nucleic acid to be expressed there. The expression is particularly influenced by the promoter or promoters which regulate the transcription. In principle, the expression may be carried out by the natural promoter originally localized before the nucleic acid to be expressed, but also by a promoter of the host cell provided on the expression vector or also by a modified or completely different promoter of another organism or another host cell. In the present case, at least one promoter is provided for the expression of a nucleic acid as contemplated herein and used for its expression. Expression vectors can also be regulated, for example by altering the cultivation conditions or when a certain cell density of the host cells they contain is reached or by adding certain substances, in particular gene expression activators. An example of such a substance is the galactose derivative isopropyl β-D-thiogalactopyranoside (IPTG), which is used as an activator of the bacterial lactose operon (lac operon). In contrast to expression vectors, the contained nucleic acid is not expressed in cloning vectors.

Another subject-matter of the present disclosure is a non-human host cell which contains a nucleic acid or vector as contemplated herein, or which contains an amylase as contemplated herein, in particular one which secretes the amylase into the medium surrounding the host cell. Preferably, a nucleic acid or vector as contemplated herein is transformed into a microorganism, which then represents a host cell as contemplated herein. Alternatively, individual components, i.e. nucleic acid parts or fragments of a nucleic acid as contemplated herein may be introduced into a host cell in such a way that the resulting host cell contains a nucleic acid or vector as contemplated herein. This procedure is particularly suitable if the host cell already contains one or several components of a nucleic acid or a vector as contemplated herein and the other components are then supplemented accordingly. Methods for the transformation of cells are established in the state of the art and sufficiently known to the expert. In principle, all cells, i.e. prokaryotic or eukaryotic cells, are suitable as host cells. Preference is given to those host cells that are genetically advantageous in terms of transformation with nucleic acid or vector and their stable establishment, for example unicellular fungi or bacteria. Furthermore, preferred host cells are exemplified by good microbiological and biotechnological manageability. This concerns, for example, easy cultivability, high growth rates, low requirements on fermentation media and good production and secretion rates for foreign proteins. Preferred host cells as contemplated herein secrete the (transgenic) expressed protein into the medium surrounding the host cells. Furthermore, the amylases may be modified by the cells producing them after their production, for example by attachment of sugar molecules, formylations, aminations, etc. Such post-translational modifications can functionally influence the amylase.

Other preferred embodiments are host cells whose activity may be regulated by genetic regulatory elements, which are provided on the vector, for example, but which may also be present in these cells from the outset. For example, the controlled addition of chemical compounds that serve as activators, by changing the cultivation conditions or when a certain cell density is reached, these may stimulate expression. This enables an economic production of the proteins as contemplated herein. An example of such a compound is IPTG as described above.

Preferred host cells are prokaryotic or bacterial cells. Bacteria are exemplified by short generation times and low demands on cultivation conditions. This allows the establishment of cost-effective cultivation methods or production methods. In addition, the expert has a wealth of experience with bacteria in fermentation technology. Gram-negative or gram-positive bacteria may be suitable for a specific production for a variety of reasons that may be determined experimentally in individual cases, such as nutrient sources, product formation rate, time requirements, etc.

In gram-negative bacteria such as Escherichia coli, a large number of proteins are secreted into the periplasmic space, i.e. into the compartment between the two membranes enclosing the cells. This may be beneficial for special applications. In addition, gram-negative bacteria may also be designed in such a way that they secrete the expressed proteins not only into the periplasmic space but also into the medium surrounding the bacterium. In contrast, gram-positive bacteria such as Bacilli or actinomycetes or other representatives of actinomycetales have no outer membrane, so that secreted proteins are immediately released into the medium surrounding the bacteria, usually the culture medium, from which the expressed proteins can be purified. They may be isolated directly from the medium or further processed. In addition, gram-positive bacteria are related or identical to most of the organisms of origin for technically important enzymes and usually form comparable enzymes themselves, so that they have a similar codon use and their protein synthesis apparatus is naturally oriented accordingly.

Host cells as contemplated herein may be altered with respect to their requirements for culture conditions, have other or additional selection markers or express other or additional proteins. In particular, these may also be host cells that transgenically express several proteins or enzymes.

The present disclosure is in principle applicable to all microorganisms, in particular to all fermentable microorganisms, especially preferably those of the genus Bacillus, and leads to the result that proteins as contemplated herein may be produced by using such microorganisms. Such microorganisms then represent host cells as contemplated herein.

In a further embodiment of the present disclosure, the host cell is a bacterium, preferably one selected from the group of the genera of Escherichia, Klebsiella, Bacillus, Staphylococcus, Corynebacterium, Arthrobacter, Streptomyces, Stenotrophomonas and Pseudomonas, further preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell may also be a eukaryotic cell, which is exemplified by the fact that it has a cell nucleus. A further subject-matter of the present disclosure is therefore a host cell which is exemplified by having a cell nucleus. In contrast to prokaryotic cells, eukaryotic cells are capable of posttranslationally modifying the protein formed. Examples are fungi such as actinomycetes or yeasts such as Saccharomyces or Kluyveromyces. This may be particularly advantageous, for example, if the proteins are to undergo specific modifications in connection with their synthesis that enable such systems. The modifications that eukaryotic systems undergo in particular in connection with protein synthesis include the binding of low-molecular compounds such as membrane anchors or oligosaccharides. Such oligosaccharide modifications may be desirable, for example, to reduce the allergenicity of an expressed protein. Co-expression with the enzymes naturally produced by such cells, such as cellulases, may also be advantageous. Furthermore, thermophilic fungal expression systems may be particularly suitable for the expression of temperature-resistant proteins or variants. In preferred embodiments of the present disclosure, the host cell is a basidiomycete cell.

The host cells as contemplated herein are cultivated and fermented in the usual way, for example in discontinuous or continuous systems. In the first case, a suitable culture medium is inoculated with the host cells and the product is harvested from the medium after a period of time to be determined experimentally. Continuous fermentations are exemplified by the achievement of flow equilibrium, in which cells partially die but also grow again over a comparatively long period of time and at the same time the protein formed can be removed from the medium.

Host cells as contemplated herein are preferably used to produce amylases as contemplated herein. A further subject-matter of the present disclosure is therefore a method for producing an amylase comprising
a) cultivating a host cell as contemplated herein, and
b) isolating the amylase from the culture medium or from the host cell.

This subject-matter of the present disclosure preferably comprises fermentation methods. Fermentation methods are per se known from the state of the art and represent the actual large-scale production step, usually followed by a suitable purification method of the manufactured product, for example, the amylase as contemplated herein. All fermentation methods based on a corresponding method for the production of an amylase as contemplated herein are embodiments of this subject-matter of the present disclosure.

Fermentation methods which are exemplified by the fact that fermentation is carried out via an inflow strategy are particularly worth considering. Here, the media components consumed by the continuous cultivation are fed in. In this way considerable increases in cell density as well as in cell mass or dry mass and/or in particular in the activity of the amylase of interest may be achieved. Furthermore, the fermentation may also be designed in such a way that undesirable metabolic products are filtered out or neutralized by the addition of buffer or appropriate counterions.

The produced amylase may be harvested from the fermentation medium. Such a fermentation method is preferred to isolation of the amylase from the host cell, i.e. product processing from the cell mass (dry mass), but requires the provision of suitable host cells or of one or several suitable secretion markers or mechanisms and/or transport systems so that the host cells secrete the amylase into the fermentation medium. In the absence of secretion, isolation of the amylase from the host cell, i.e. purification of the amylase from the cell mass, may alternatively be achieved, for example by precipitation with ammonium sulfate or ethanol, or by chromatographic purification.

All of the above facts may be combined to form a method for producing amylases as contemplated herein.

Another subject-matter of the present disclosure is a product exemplified by the fact that it contains an amylase as described herein. The amylase thereby exhibits about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8% or about 99.9% sequence identity with the amino acid sequence indicated in SEQ ID NO. 1 or SEQ ID NO. 2 over its entire length, or is a variant thereof as described above, which is obtainable starting from an amylase described above as the starting molecule and has the indicated sequence identity with SEQ ID NO. 1 or SEQ ID NO. 2 before and preferably also after the variation. Preferably the product is a detergent or cleaning agent.

This subject-matter of the present disclosure includes all conceivable types of detergents or cleaning agents, both concentrates and undiluted, for use on a commercial scale, in washing machines or for hand washing or cleaning. This includes, for example, detergents for fabrics, carpets or natural fibers, for which the term detergent is used. It also includes, for example, dishwashing detergents for dishwashers or manual dishwashing detergents or cleaners for hard surfaces such as metal, glass, porcelain, ceramics, tiles, stone, painted surfaces, plastics, wood or leather, for which the term detergent is used, i.e. in addition to manual and machine dishwashing detergents, for example, also scouring agents, glass cleaners, toilet air fresheners, etc. The detergents and cleaning agents within the scope of the present disclosure also include washing auxiliary agents which are added to the actual washing agent during manual or machine fabric washing in order to achieve a further effect. Furthermore, detergents and cleaning agents within the scope of the present disclosure also include fabric pre- and post-treatment products, i.e. those products with which the laundry item is brought into contact before the actual washing, for example to dissolve stubborn stains, and also those products which, in a step downstream of the actual fabric washing, give the laundry item further desirable properties such as a pleasant feel, freedom from creasing or low static charge. Among the latter products are fabric softeners.

The detergents or cleaning agents as contemplated herein, which may be in the form of powdery solids, in post-compressed particle form, as homogeneous solutions or suspensions, may contain, in addition to an amylase as contemplated herein, all known and customary ingredients in such agents, preferably at least one further ingredient being present in the product. The products as contemplated herein may in particular contain surfactants, builders, peroxygen compounds or bleach activators. Furthermore, they may contain water-miscible organic solvents, further enzymes, sequestering agents, electrolytes, pH regulators and/or further auxiliary substances such as optical brighteners, graying inhibitors, foam regulators as well as dyes and fragrances as well as combinations thereof.

In particular, a combination of an amylase as contemplated herein with one or several further ingredient(s) of the product is advantageous, since such a product in preferred embodiments as contemplated herein has an improved cleaning performance due to the resulting synergisms. Such a synergism may be achieved in particular by combining an amylase as contemplated herein with a surfactant and/or a builder and/or a peroxygen compound and/or a bleach activator.

Advantageous ingredients of products as contemplated herein are disclosed in the international patent application WO 2009/121725, there beginning on page 5, penultimate paragraph, and ending on page 13 after the second paragraph. Express reference is made to this disclosure and the disclosure content there is included in the present patent application.

In further embodiments of the present disclosure, the product contains
(a) from about 1 to about 85% by weight, preferably from about 5 to about 65% by weight of surfactants; and/or
(b) from about 0 to about 45% by weight, preferably from about 0.1 to about 15% by weight of builders; and/or
(c) from about 0.0005 to about 15% by weight, preferably from about 0.001 to about 5% by weight, of protease; and/or
(d) from about 0.0005 to about 15% by weight, preferably from about 0.001 to about 5% by weight, of lipase; and/or
(e) from about 0.00005 to about 15% by weight, preferably from about 0.0001 to about 5% by weight, of mannanase; and/or
(f) from about 0.00005 to about 15% by weight, preferably from about 0.0001 to about 5% by weight, cellulase/pectate lyase; and/or
(g) from about 0.00005 to about 15 g/wash load, preferably from about 0.0001 to about 5 g/wash load, of xanthan lyase; and/or
(h) from about 0.00005 to about 15 g/wash load, preferably from about 0.00005 to about 15 g/wash load, endoglucanase capable of digesting xanthan gum.

A product as contemplated herein contains the amylase advantageously in an amount of from about 2 μg to about 20 mg, preferably from about 5 μg to about 17.5 mg, particularly preferably from about 20 μg to about 15 mg and very particularly preferably from about 50 μg to about 10 mg per gram of the product. In addition, the product as contemplated herein may advantageously contain the amylase in an amount from about 0.00005 to about 15% by weight with respect to the active enzyme and the total weight of the product, preferably from about 0.0001 to about 5% by weight and particularly preferably from about 0.001 to about 1% by weight. In addition, the amylase contained in the product, and/or other ingredients of the product, may be coated with a substance which is impermeable by the enzyme at room temperature or in the absence of water and which becomes permeable by the enzyme under the conditions of use of the product. Such an embodiment of the present disclosure is thus exemplified in that the amylase is coated with a substance which is impermeable by the amylase at room temperature or in the absence of water. Furthermore, the detergent or cleaning agent itself may also be packaged in a container, preferably an air-permeable container, from which it is released shortly before use or during the washing process.

In further embodiments of the present disclosure the product is
a) in solid form, in particular in the form of a free-flowing powder with a bulk density of from about 300 g/l to about 1200 g/l, in particular from about 500 g/l to about 900 g/l; or
b) in paste or liquid form; and/or
c) in the form of a gel or a sachet (pouches); and/or
d) a single component system; or
e) divided into several components.

These embodiments of the present disclosure comprise all solid, powdery, liquid, gel or pasty dosage forms of products as contemplated herein, which may also consist of several phases and may be in compressed or non-compressed form. The product may be present as a free-flowing powder, especially with a bulk density of from about 300 g/l to about 1200 g/l, especially from about 500 g/l to about 900 g/l or from about 600 g/l to about 850 g/l. The solid dosage forms of the product also include extrudates, granules, tablets or pouches. Alternatively, the product may also be liquid, gel or paste, for example in the form of a non-aqueous liquid detergent or non-aqueous paste or in the form of an aqueous liquid detergent or aqueous paste. Furthermore, the product may be in the form of a one-component system. Such products consist of one phase. Alternatively, a product may also consist of several phases. Such a product is therefore divided into several components.

Detergents and cleaning products as contemplated herein may only contain one amylase. Alternatively, they may also contain other hydrolytic enzymes or other enzymes in a concentration appropriate for the effectiveness of the product. Products which further comprise one or several further enzymes thus represent a further embodiment of the present disclosure. Preferably usable as further enzymes are all enzymes which may develop a catalytic activity in the product as contemplated herein, in particular a protease, lipase, cellulase, hemicellulase, mannanase, tannase, xylanase, xanthanase, xyloglucanase, β-glucosidase, pectinase, carrageenase, perhydrolase, oxidase, oxidoreductase or other amylases—distinguishable from the amylases as contemplated herein—as well as mixtures thereof. Further enzymes are advantageously contained in the product in an amount of about 1×10⁻⁸ to 5% by weight, based on active protein. Increasingly preferably, each further enzyme is contained in the products as contemplated herein in an amount of about 1×10⁻⁷ to 3% by weight, of from about 0.00001 to about 1% by weight, of from about 0.00005 to about 0.5% by weight, of from about 0.0001 to about 0.1% by weight and particularly preferably of from about 0.0001 to about 0.05% by weight, based on active protein. The enzymes show particularly preferential synergistic cleaning performance in relation to certain soiling or stains, i.e. the enzymes contained in the product composition support each other in their cleaning performance. Such synergism is particularly preferred between the amylase contained in the product composition as contemplated herein and another enzyme of a product as contemplated herein, including in particular between the said amylase and a lipase and/or a protease and/or a mannanase and/or a cellulase and/or a pectinase. Synergistic effects may occur not only between different enzymes, but also between one or several enzymes and further ingredients of the product as contemplated herein.

In the cleaning products described herein, the enzymes to be used may also be packaged together with accompanying substances, for example from fermentation. In liquid formulations the enzymes are preferably used as enzyme liquid formulation(s).

The enzymes are usually not provided in the form of the pure protein, but rather in the form of stabilized preparations suitable for storage and transport. These pre-packaged preparations include, for example, solid preparations obtained by granulation, extrusion or lyophilization or, especially in the case of liquid or gel products, solutions of the enzymes, advantageously as concentrated as possible, low in water and/or mixed with stabilizers or other auxiliary substances.

Alternatively, the enzymes may be encapsulated for both solid and liquid dosage forms, for example by spray-drying or extrusion of the enzyme solution together with a preferably natural polymer, or in the form of capsules, for example those in which the enzymes are enclosed as in a solidified gel or in core-shell type capsules in which an enzyme-containing core is covered with a protective layer impermeable by water, air and/or chemicals. Additional active ingredients, such as stabilizers, emulsifiers, pigments, bleaching agents or dyes, may also be applied in superimposed layers. Such capsules are applied by methods known per se, for example by shaking or rolling granulation or in fluid-bed processes. Advantageously, such granulates, for example by applying polymer film formers, are low-dust and, due to the coating, stable in storage.

The enzymes may also be inserted into water-soluble films. Such a film allows the enzymes to be released after contact with water. As used here, “water-soluble” refers to a film structure that is preferably completely water-soluble. However, films which are essentially water soluble but contain relatively small amounts of a material in the film structure which is not water soluble, films containing materials which are water soluble only at relatively high water temperatures or only under limited pH conditions, and films which include a relatively thin layer of water-insoluble material are all included in the term “water soluble”. Preferably such a film including (fully or partially hydrolyzed) polyvinyl alcohol (PVA). However, the film may also contain, exclusively or in addition to PVA, acid/acrylate copolymers, preferably methacrylic acid/ethyl acrylate copolymer, such as that available from Belland as GBC 2580 and 2600, styrene-maleic anhydride copolymer (SMA) (available as Scripset (trade name) from Monsanto), ethylene-acrylic acid copolymer (EAA) or metal salt-neutralized ethylene-methacrylic acid copolymer (EMAA) known as Ionomer (available from DuPont) in which the acid content of EAA or EMAA is at least about 20 mole percent, polyether block amide copolymer, polyhydroxyvaleric acid (available as Imperial Chemical Industries' Biopol (trade name) resins), polyethylene oxide, water-soluble polyester or copolyester, polyethyloxazoline (PEOX 200 from Dow), and water-soluble polyurethane.

Furthermore, it is possible to combine two or more enzymes together, so that a single granule exhibits several enzyme activities.

A further subject-matter of the present disclosure is a method for cleaning fabrics or hard surfaces, which is exemplified in that in at least one method step a product as contemplated herein is used, or that in at least one method step an amylase as contemplated herein becomes catalytically active, in particular in such a way that the amylase is used in an amount of from about 40 μg to about 4 g, preferably of from about 50 μg to about 3 g, particularly preferably of from about 100 μg to about 2 g and very particularly preferably of from about 200 μg to about 1 g.

In various embodiments, the method described above is exemplified by the use of amylase at a temperature of from about 0 to about 100° C., preferably from about 0 to about 60° C., further preferably from about 20 to about 45° C. and most preferably about 40° C.

This includes both manual and mechanical methods, with mechanical methods being preferred. Methods for cleaning fabrics are generally exemplified by the fact that in several method steps different active cleaning substances are applied to the material to be cleaned and washed off after the exposure time, or that the material to be cleaned is otherwise treated with a detergent or a solution or dilution of this product. The same applies to methods for cleaning all materials other than fabrics, especially hard surfaces. All conceivable washing or cleaning methods may be enriched in at least one of the method steps by the application of a detergent and cleaning product or an amylase as contemplated herein and then constitute embodiments of the present disclosure. All facts, subject-matters and embodiments described for amylases and products containing them as contemplated herein are also applicable to the subject-matter of this present disclosure. Therefore, explicit reference is made to the disclosure at the appropriate place with the remark that this disclosure also applies to the above method as contemplated herein.

Since amylases as contemplated herein naturally already possess a hydrolytic activity and also develop this activity in media which otherwise have no cleaning power such as, for example, in a mere buffer, a single and/or the only step of such a method may consist in bringing, if desired, an amylase as contemplated herein into contact with the soiled material as the only cleaning active component, preferably in a buffer solution or in water. This represents a further embodiment of the subject-matter of the present disclosure.

Alternative embodiments of the subject-matter of the present disclosure also represent methods for the treatment of fabric raw materials or for fabric care, in which an amylase as contemplated herein becomes active in at least one method step. Methods for fabric raw materials, fibers or fabrics with natural components are preferred, and especially for those with wool or silk.

In a further aspect, the present disclosure relates to the use of an amylase as contemplated herein or an amylase available according to a method as contemplated herein in a detergent and cleaning product for the removal of starch-containing stains.

All facts, subject-matters and embodiments described for the amylase and products containing it as contemplated herein are also applicable to the described method and uses. Therefore, explicit reference is made to the disclosure at the appropriate place with the remark that this disclosure also applies to the above-mentioned uses and methods as contemplated herein.

In a further aspect, the present disclosure relates to the use of an amylase as contemplated herein or an amylase obtainable by a method as contemplated herein or an amylase used as described in the products of the present disclosure in a detergent and cleaning product for the removal of starch-containing stains. All facts, subject-matters and embodiments described for amylase as contemplated herein and products containing it are also applicable to this subject-matter as contemplated herein.

### EXAMPLES

### Example 1

### Identification of Amylase

It was screened in basidiomycetes for starch-degrading enzymes. A wild-type enzyme, annotated as alpha-amylase, from Fomitopsis pinicola (Fpi) was detected. This amylase showed good washing performance on various starch-containing fabrics.

The sequence of the amylase found in Fomitopsis pinicola differs significantly from the sequences of amylases previously used in detergents and cleaning products. This opens up many options for increasing the genetic and biochemical diversity of amylases used in cleaning products.

### Example 2

### Screening for Amylase with Washing Performance

Cultivation and Concentration

Fomitopsis pinicola was cultivated in a standard liquid nutrient (SNL) medium, with glucose replaced by 1% soluble starch as a carbon source. The presence of starch in the medium was checked by means of Lugol's solution. To this end, 20 μl Lugol's solution (Sigma) was added to 200 μl culture supernatant. When no more starch was detected in the medium, the cultivation was stopped. After cultivation for nine days, the culture supernatant was concentrated by means of Centricons® Plus-70 membranes (Merck Milipore, Darmstadt; MWCO 10 kDa) to increase the enzyme concentration. Centrifugation, which was performed at 4000 g and 4° C., was stopped as soon as the liquid flow stopped.

### Example 3

### Activity Assay for Amylase

A modified para-nitrophenyl maltoheptaoside whose terminal glucose unit was blocked by a benzylidene group was used to determine the amylolytic activity of amylases as contemplated herein. From this molecule, the amylase releases para-nitrophenyl oligosaccharide, which in turn is converted into glucose and para-nitrophenol by the enzymes glucoamylase and alpha-glucosidase. Thus the amount of para-nitrophenol released is proportional to the activity of the amylase. The measurement is performed, for example, with the aid of the Quick-Start® test kit from Abbott (Abbott Park, Ill., USA). The increase in absorbance (405 nm) in the test kit was determined at 37° C. for 3 minutes compared to a photometric control value (blank value). The calibration was performed on an enzyme standard with known activity (e.g. Maxamyl®/Purastar® 2900 Genencor 2900 TAU/g). The evaluation was performed by determining the absorbance difference dE (405 nm) per minute against the enzyme concentration of the standard.

### Example 4

### Wash Test and Results

A wash test was performed with the purified wild-type supernatant from Fomitopsis pinicola containing the described amylase.

Conditions: 40° C., 16° dH water, 1 h;

Enzyme concentration: 0.186 TAU/ml (determination of amylase activity with benzylidene blocked para-nitrophenol maltoheptaoside); this corresponds to the amount of amylase normally used in detergents.

Stains:

1. C-S-27 potato starch (from CFT)

2. 10R Starch/soot (from WFK)

Execution:
Place punched fabric (diameter=10 mm) in a microtiter plate, preheat wash solution to 40° C., final concentration 4.58 g/L;
Add basic solution and enzyme to the stain, incubate for 1 h at 40° C. and 600 rpm;
then rinse the stain several times with clear water, leave to dry and determine the brightness with a colorimeter.

The brighter the fabric becomes, the better the cleaning performance Here, the L-value=brightness is measured, the higher the brighter.

It is washed with a common liquid detergent without enzymes (see table 2).

Sample 1: Only detergents as benchmark (comparative reference)

Sample 2: Detergents plus amylase from Fomitopsis pinicola (according to SEQ ID NO. 1)

The results are presented in Table 1.

TABLE 1
Results of the wash tests

| Stain | Sample 1 | Sample 2 |
|---|---|---|
| Potato starch | 75.4 | 79.8 |
| Starch/soot | 66.4 | 68.5 |

It is clear that the amylase on both stains causes an improved performance of the detergent. As a negative control, the boiled, purified supernatant from Fomitopsis pinicola (99° C. for 30 min) was washed along with the stains, but it did not show any washing performance (not shown).

TABLE 2
Detergent matrix used

| | % by weight active | % by weight active |
|---|---|---|
| | substance in raw | substance in |
| Chemical name | material | formulation |
| Water demin. | 100 | Remainder |
| Alkylbenzene sulfonic | 96 | 4.40 |
| acid | | |
| Other anionic surfactants | 70 | 5.60 |
| C₁₂-C₁₈ fatty acids Na salt | 30 | 2.40 |
| Non-ionic surfactants | 100 | 4.40 |
| Phosphonates | 40 | 0.20 |
| Citric acid | 100 | 1.40 |
| NaOH | 50 | 0.95 |
| Defoamer | t.q. | 0.01 |
| Glycerin | 100 | 2.00 |
| Preservation products | 100 | 0.08 |
| Ethanol | 93 | 1.00 |

The matrix used contained no optical brighteners, perfumes, dyes or enzymes.
Dosage: 4.58 g/L

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An amylase comprising an amino acid sequence having at least about 70% sequence identity to SEQ ID NO. 1 or SEQ ID NO. 2 over its entire length.

2. The amylase according to claim 1, wherein the amylase is obtained from
a) first starting amylase as a first starting molecule by single or multiple conservative amino acid substitution; and/or
b) a second starting amylase as a second starting molecule by fragmentation, deletion, insertion or substitution mutagenesis wherein the amino acid sequence of the amylase is identical to the amino acid sequence of the starting amylase over a length of at least about 403 contiguous amino acids.

3. A method for producing the amylase according to claim 1, the method comprising providing a starting amylase having at least about 70% sequence identity to SEQ ID NO. 1 or SEQ ID NO. 2 over its entire length.

4. The method according to claim 3, wherein the method further comprises one or both of the following method steps:
a) introducing a single or multiple conservative amino acid substitution into the starting amylase to form a first variant, wherein the first variant has at least about 70% sequence identity to SEQ ID NO. 1 or SEQ ID NO. 2 over its entire length;
b) altering the amino acid sequence of the starting amylase by fragmentation, deletion, insertion or substitution mutagenesis to form a second variant, wherein the second variant comprises an amino acid sequence which, over a length of at least 403, contiguous amino acids is identical to the starting amylase.

5. The acid encoding an amylase according to claim 1, wherein a nucleic acid encodes for the amylase.

6. The amylase according to claim 5, wherein a vector comprises the nucleic acid.

7. The amylase according to claim 1, wherein a non-human host cell comprises the amylase, and wherein the non-human host cell secretes the amylase into a medium surrounding the non-human host cell.

8. (canceled)

9. A detergent and cleaning product, wherein the detergent and cleaning product comprises at least one amylase according to claim 1, wherein the at least one amylase is comprised in the detergent and cleaning product in an amount of from about 0.00005 to about 15% by weight based on active protein.

10. (canceled)

11. (canceled)

12. The amylase according to claim 1, wherein the amino acid sequence has at least about 70% sequence identity to SEQ ID NO. 1 over its entire length, wherein the amylase has at least one substitution, deletion, and/or insertion compared to SEQ ID NO. 1, and wherein the amylase has enzymatic activity.

13. The amylase according to claim 12, wherein the amino acid sequence has at least about 80% sequence identity to SEQ ID NO. 1 over its entire length.

14. The amylase according to claim 13, wherein the amino acid sequence has at least about 90% sequence identity to SEQ ID NO. 1 over its entire length.

15. The amylase according to claim 14, wherein the amino acid sequence has at least 95% sequence identity to SEQ ID NO. 1 over its entire length.

16. The amylase according to claim 15, wherein the amino acid sequence has at least 99% sequence identity to SEQ ID NO. 2 over its entire length.

17. The amylase according to claim 1, wherein the amino acid sequence has at least about 70% sequence identity to SEQ ID NO. 2 over its entire length, wherein the amylase has at least one substitution, deletion, and/or insertion compared to SEQ ID NO. 2, and wherein the amylase has enzymatic activity.

18. The amylase according to claim 17, wherein the amino acid sequence has at least about 80% sequence identity to SEQ ID NO. 2 over its entire length.

19. The amylase according to claim 18, wherein the amino acid sequence has at least about 90% sequence identity to SEQ ID NO. 2 over its entire length.

20. The amylase according to claim 19, wherein the amino acid sequence has at least 95% sequence identity to SEQ ID NO. 2 over its entire length.

21. The amylase according to claim 20, wherein the amino acid sequence has at least 99% sequence identity to SEQ ID NO. 2 over its entire length.

22. The amylase according to claim 6, wherein the vector is a cloning vector.

23. The amylase according to claim 6, wherein the vector is an expression vector.
